(19) United States
(12) Patent Application Publication
(10) Pub. No.: US 2023/0069394 A1
(43) Pub. Date: Mar. 2, 2023

(54) TATTOO PEN SYSTEMS AND METHODS

(71) Applicant: Carlos Romero, Moreno Valley, CA (US)

(72) Inventor: Carlos Romero, Moreno Valley, CA (US)

(21) Appl. No.: 17/820,888

(22) Filed: Aug. 19, 2022

Related U.S. Application Data
(60) Provisional application No. 63/240,060, filed on Sep. 2, 2021.

(51) Int. Cl. G09B 19/24

(52) U.S. Cl. CPC G09B 19/24; A61M 37/0076

(57) ABSTRACT

A tattoo pen system is claimed, where a pen base may be configured to mate with a plurality of pen heads to allow for a user to freely interchange one head for another during use. A practice pen having interchangeable heads may be sized and shaped to have similar dimensions and weight to a mechanized pen having interchangeable heads. This allows for a user to practice drawing stencils and applying tattoos using a practice pen base with interchangeable heads, and then later smoothly transition to drawing stencils and applying tattoos using a mechanized pen base with interchangeable heads. The pen base of either the practice pen or the mechanized pen may be configured to mate with a plurality of needle cartridges.

### CROSS-REFERENCE TO RELATED APPLICATIONS

This application claims the benefits of U.S. Prov. Ser. No. 63/240,060, filed on 2021 Sep. 2, the entire contents of which is expressly incorporated herein by reference.

### STATEMENT RE: FEDERALLY SPONSORED RESEARCH/DEVELOPMENT

Not Applicable.

### BACKGROUND

To apply a tattoo to a person's skin, a tattoo artist typically first applies a stencil to the person's skin and then applies ink to the lines of the stencil using a tattoo needle that breaks the person's skin. When the skin breaks, the person bleeds, and the blood has to be removed by lightly pressing on the skin with a cloth. During this process, the stencil may smear or otherwise come off of the skin. This is problematic because the tattoo artist then no longer has the lines to follow to draw the artwork on the person. The process of restencilling the artwork on the person is problematic.

Accordingly, there is a need in the art for improving the equipment and the process of tattooing.

### BRIEF SUMMARY

Tattoo pen systems and methods are disclosed herein.

The tattoo pen system discussed herein may include a practice pen with a practice pen base and/or mechanized pens with a mechanized pen base. A mechanized pen base may have a motor that is configured to repeatedly advance and retract a needle tip to apply ink to a person's skin, while a practice pen base may have a motor that is configured to vibrate and simulate the feel of a mechanized pen motor. A practice pen base may be used to practice applying ink to a practice surface, such as a piece of paper, using a pen head that attaches to the practice pen base, while a mechanized pen base may be used to apply ink to a person's skin using a pen head or a needle cartridge that attaches to the mechanized pen base. The practice pen base may have a shape, size, and/or weight similar to the shape, size, and/or weight of the mechanized tattoo pen. For example, the pen base of both the practice pen and the mechanized pen may have weights that are both within 10 grams of one another, and/or the pen base of both the practice pen and the mechanized pen may have widths that are both within 5 mm of one another. The weight of the practice pen and the mechanized pen may be at least 100 grams, and/or the width of the practice pen and the meachanized pen may be at least 2 cm. Using a practice pen base that has a similar shape, size, and/or weight to the mechanized pen base allows for a user of the practice pen base to easily build muscle memory while practicing with the practice pen base for some time. When a user who has been practicing with the practice pen base to draw a tattoo stencil on a practice surface later uses the mechanized pen base to draw a tattoo stencil on a person's skin, the user rapidly adjusts to using the mechanized pen base.

A tattoo pen may have a pen base having a grip and a cavity shaped to accept a shaft of a pen head and/or a needle cartridge that plugs into the cavity of the pen base. Each pen head may have a pen tip that is configured to absorb a volume of ink, for example at least 0.2 ml of ink. The pen tips of the pen heads may differ from one another, for example one pen tip may comprise a porous foam similar to a Sharpie® tip whereas another pen tip may comprise a brush similar to a paintbrush. A set of tattoo pens may comprise both a practice pen base and a mechanized pen base having interchangeable pen heads and needle cartridges configured to mate with either the practice pen base or the mechanized pen base to allow a user to first practice applying ink to a surface using the practice pen, and then to apply ink to a surface using the mechanized pen.

When utilizing a mechanized tattoo pen, a user may apply ink to a stencil on a person's skin using a needle of the tattoo pen, but may need to redraw the stencil if the user accidentally wipes a portion of the stencil away while trying to wipe away blood. The user may then remove the needle cartridge containing the needle from the pen base of the tattoo pen, and then may attach a pen head to the pen base. The user may then draw on the stencil using the pen head before removing the pen head and attaching a fresh needle cartridge to continue tattooing along the stencil using the needle of the needle cartridge. A plurality of interchangeable pen heads may be provided, allowing a user to draw or redraw a stencil using a first pen head, and then switch the pen head with another pen head when the user needs a fresh pen head—for example if the first pen head becomes adulterated with a customer's blood.

The interchangeable pen heads may all be identical, or some of the pen heads may have different style tips, such as a sharp tip, a blunt tip, and a brush tip. In this manner, the user may use the same pen to apply ink to a stencil using a needle, and then redraw the stencil using the same pen using a variety of different tip styles or even different colors of ink. If the user needs to use a particular color ink, the user may simply dip the tip of the pen head into an inkwell to absorb ink, and then may draw on the stencil using the tip of the pen head. If the user wishes to tattoo ink to a person's skin, the user may remove a pen head from a mechanized pen base, and may attach a needle cartridge to the mechanized pen base. The user may then activate a motor in the pen base to repeatedly advance and retract the needle in the needle cartridge. Different pen heads may be attached to the pen base by removing the current pen head or needle cartridge from the pen base, and then by attaching a different pen head to the pen base, allowing a user to choose between different sizes or types of pen tips while using the same pen base.

### BRIEF DESCRIPTION OF THE DRAWINGS

These and other features and advantages of the various embodiments disclosed herein will be better understood with respect to the following description and drawings, in which like numbers refer to like parts throughout, and in which:

FIG. 1 shows side plan view of a practice tattoo pen.

FIG. 2 shows a side perspective view of the practice tattoo pen of FIG. 1.

FIG. 3 shows an exploded view of the practice tattoo pen of FIG. 1, with four different types of pen heads with the caps removed.

FIG. 4 shows an exploded view of a mechanized tattoo pen, with five different types of pen heads with the caps added to the pen heads.

FIG. 5 shows a side perspective view of a pen kit having a plurality of pen heads, a plurality of ink wells, and an ink canister.

FIG. 6 shows a top perspective view of an ink well of FIG. 5.

FIG. 7 shows a side perspective view of the ink canister of FIG. 5.

FIG. 8 shows a top perspective view of the ink canister of FIG. 7 disposing of ink into the ink well of FIG. 6.

FIG. 9 shows a top perspective view of the practice pen of FIG. 1 with the cap removed.

FIG. 10 shows a top perspective view of the practice pen of FIG. 9 with the ink tip dipped into the ink well of FIG. 8.

FIG. 11 shows a top perspective view of the practice pen of FIG. 9 with the ink tip partially saturated with ink.

FIG. 12 shows a top perspective view of the practice pen of FIG. 11 being used to apply ink to a surface.

FIG. 13 shows a top perspective view of the mechanized pen of FIG. 4 being used to apply ink to a surface.

### DETAILED DESCRIPTION

The following detailed description describes a novel tattoo pen system and methods to use such systems.

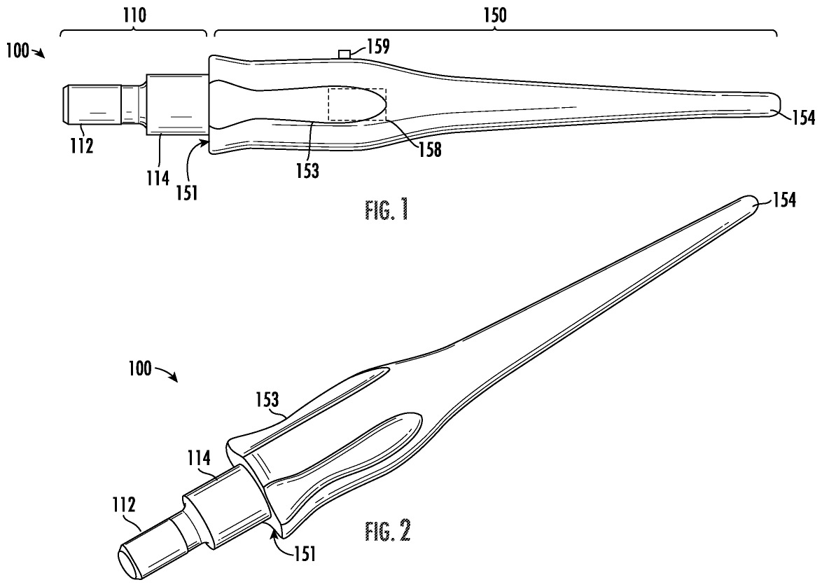

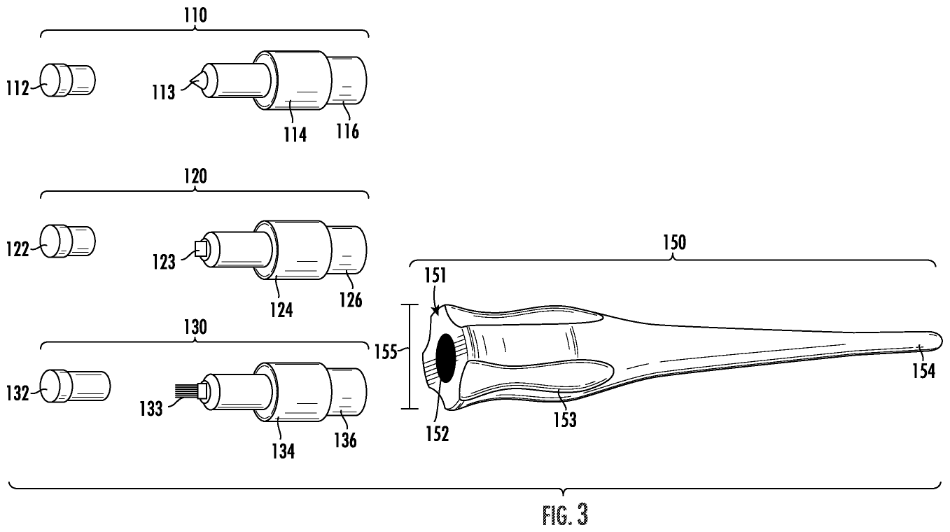

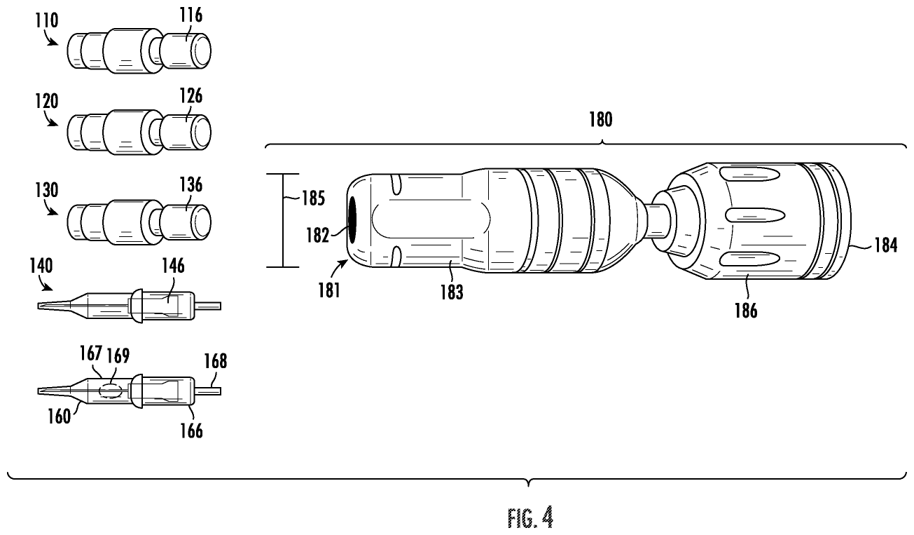

Various tattoo pen systems may be used to apply ink to a stencil or to the skin of a user via a needle. A practice tattoo pen 100 is shown in FIG. 1, having a practice pen base 150 and a pen head 110. The practice tattoo pen 100 may be used to apply ink to a practice surface, such as a piece of paper or a person's skin. Since the pen head 110 in FIG. 1 may be detached from the practice pen base 150, as shown in FIG. 3, the pen head 110 may be interchangeable with other pen heads. The pen heads may be identical to one another. For example multiple identical pen heads 110 and 170 shown in FIG. 3 may be provided to allow a user to use the same drawing motion and style on a plurality of customers, exchanging one pen head for another, identical pen head. The pen heads may also vary from one another. For example, the pen heads 110, 120, and 130 shown in FIG. 3 may be interchangeable with one another. As shown in FIG. 3, each of the pen heads 110, 120, 130, and 170 may have a shaft 116, 126, 136, and 176, respectively. Each of the shafts 116, 126, 136, and 176 may mate with the cavity 152 of the practice pen base 150 to form the practice tattoo pen 100 shown in FIG. 1. As shown in FIG. 3, each of the pen heads 110, 120, 130, and 170 may have a pen tip 113, 123, 133, and 170, respectively, which may be covered by a pen cap 112, 122, 132, and 172, respectively. Each of the pen tips 113, 123, and 133 may be configured to be different from one another, or the pen tips 113 and 173 may be configured to be identical to one another.

Regarding pen tips that are different from one another, as shown in FIG. 3, the pen heads 110, 170 may both comprise a porous foam tip 113, 173 shaped to have a sharpened tip used to apply thin ink lines to a person's skin. The pen head 120 may comprise a porous foam tip 123 extending from a body 124 shaped to have a dull tip that may be used to apply thick ink lines to the person's skin. The pen head 133 may comprise a brush tip 133 extending from a body 134 shaped to apply ink to the person's skin using stylized brush strokes. Other configurations of the tip are also contemplated. It is contemplated that a user of the practice tattoo pen 100 may switch one pen head for another depending upon the situation.

The practice tattoo pen 100 may also have a motor 158 (see FIG. 1) that is configured to make the practice tattoo pen 100 vibrate, which helps to simulate the feel of a motor in a mechanized tattoo pen. A user may activate the motor 158 by twisting a portion of the practice pen base 150 as is done on a mechanized pen base 180 (see FIG. 4), or by pressing on the button 159 (see FIG. 1), and may deactivate the motor 158 by twisting the portion of the practice pen base 150 in the opposite direction, or by pressing on the same button 159.

Each of the shafts 116, 126, 136, or 176 shown in FIG. 3 may also be shaped to mate with a mechanized pen base, such as the mechanized pen base 180 (e.g., tattoo pen) shown in FIG. 4. The mechanized pen base 180 may have a cavity 182 defined by an inner circumference and a motor 186, which may be configured to repeatedly advance and retract a needle, such as the needle tip 143 shown in FIG. 3. A user may activate the motor 186 by twisting the motor 186 relative to the grip 183 in one direction, and may deactivate the motor 186 by twisting the motor 186 relative to the grip 183 in an opposing direction. Alternatively, the user may activate and deactivate the motor 186 by pressing on a button, such as the button 159 of the practice pen base 150 (see FIG. 3). The mechanized pen 180 of FIG. 4 may normally be configured to mate with a standard tattoo needle cartridge, such as the tattoo needle cartridges 140 and. However, a user may alternatively install any of the pen heads 110, 120, 130, or 170 into the cavity 182. For example, during use, a user may follow a stencil using the needle of the tattoo needle cartridge 160 and the mechanized pen base 180. Periodically, the tattoo artist must wipe the blood off of the person's skin. In doing so, the tattoo artist may accidentally wipe part of the stencil off of a customer's skin. The tattoo artist may then remove the tattoo pen needle 160 from the mechanized pen base 180 from the cavity 182, and may then insert the pen head 110 into the cavity 182 to draw the stencil back onto the user. If the pen head 110 is then contaminated with the customer's blood, the user may then remove the pen head 110 and throw it away, replacing it with a new, sterile pen head, such as the pen head 170, at any time.

The practice pen base 150 of FIG. 3 and the mechanized pen base 180 of FIG. 4 may have similar features, such that a user who practices with the practice pen base 150 does not feel any significant difference when switching between the practice pen base 150 and the mechanized pen base 180. For example, the width and the weight of the practice pen base 150 of FIG. 3 and the mechanized pen base 180 of FIG. 4 may be configured to be similar to one another. Specifically, as shown in FIG. 3, the practice pen base 150 may have a width 155 about the grip 153 of the practice pen base 150. The width 155 of the practice pen base 150 shown in FIG. 3 may be similar to the width 185 about the grip 183 of the mechanized pen base 180 shown in FIG. 4. For example the width 155 of FIG. 3 and the width 185 of FIG. 4 may be within 5 mm of one another, and/or the width 155 of FIG. 3 and the width 185 of FIG. 4 may both be at least 2 cm. The weight of the practice pen base 150 of FIG. 3 and the mechanized pen base 180 of FIG. 4 may also be similar to one another. For example, the weight of the practice pen base 150 of FIG. 3 and the mechanized pen base 180 of FIG. 4 may be within 10 grams from one another, and/or the weight of the practice pen base 150 of FIG. 3 and the mechanized pen base 180 of FIG. 4 may be at least 100 grams. By configuring the practice pen base 150 of FIG. 3 and the mechanized pen base 180 of FIG. 4 to have similar widths and/or similar weights to one another, a user using the practice pen base 150 of FIG. 3 may develop a muscle memory when practicing with the practice pen base 150 such that there is a smooth transition when the user changes from using the practice pen base 150 to using the mechanized pen base 180 of FIG. 4.

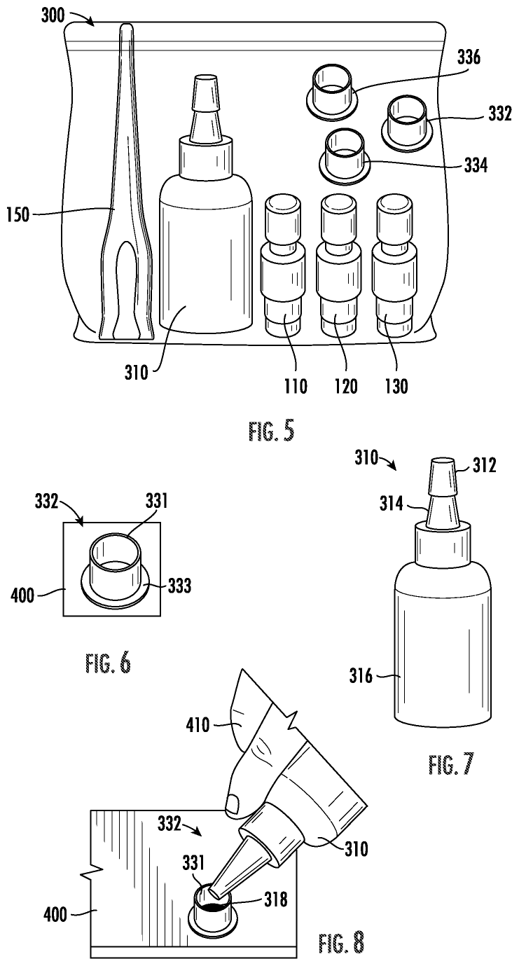

Parts of a practice tattoo pen may be sold to, or otherwise provided to, a user in a kit. For example, in FIG. 5, a plastic bag 300 may be provided containing a practice pen base 150, an ink bottle 310, a set of pen heads 110, 120, and 130, and a set of inkwells 332, 334, and 336. The set of pen heads 110, 120, and 130 may interchangeably be coupled to the practice pen base 150, and the ink bottle 310 may be used to deposit ink into cavities of the set of inkwells 332, 334, and 336, respectively. A close-up view of the inkwell 332 of FIG. 5 is shown in FIG. 6, while a close-up view of the ink bottle 310 of FIG. 5 is shown in FIG. 7.

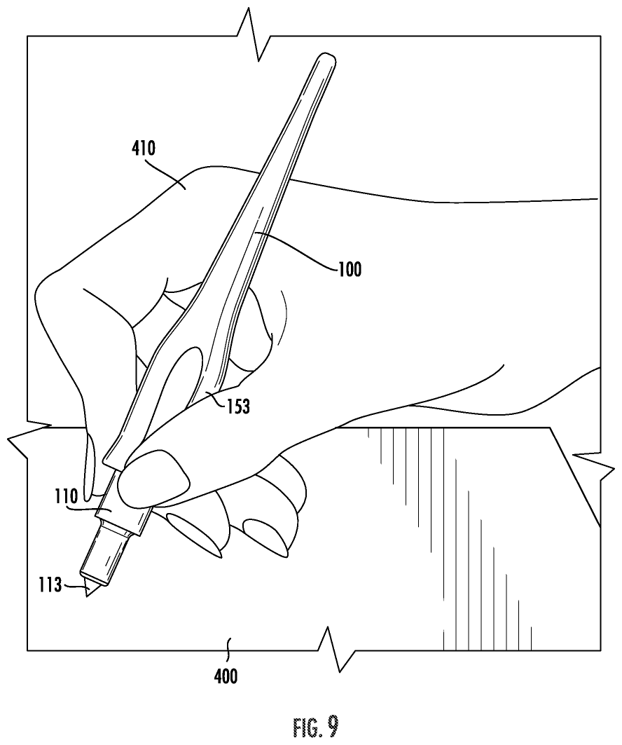

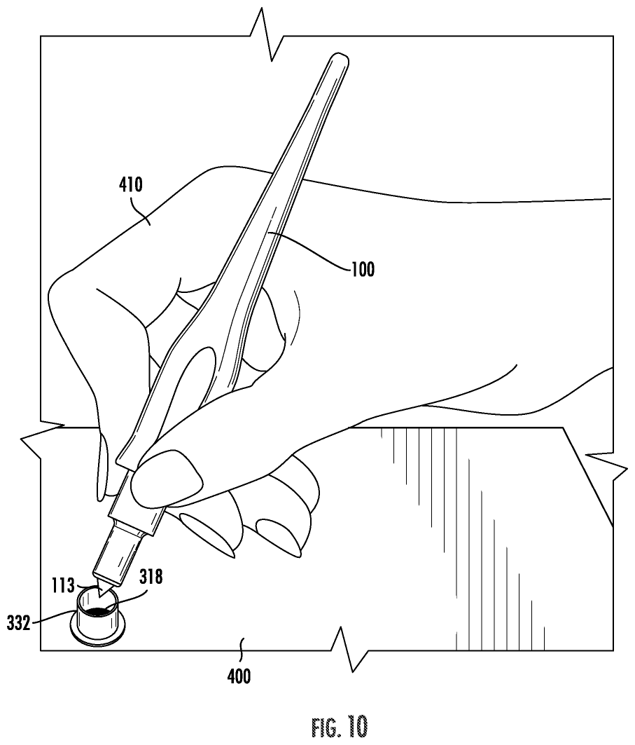

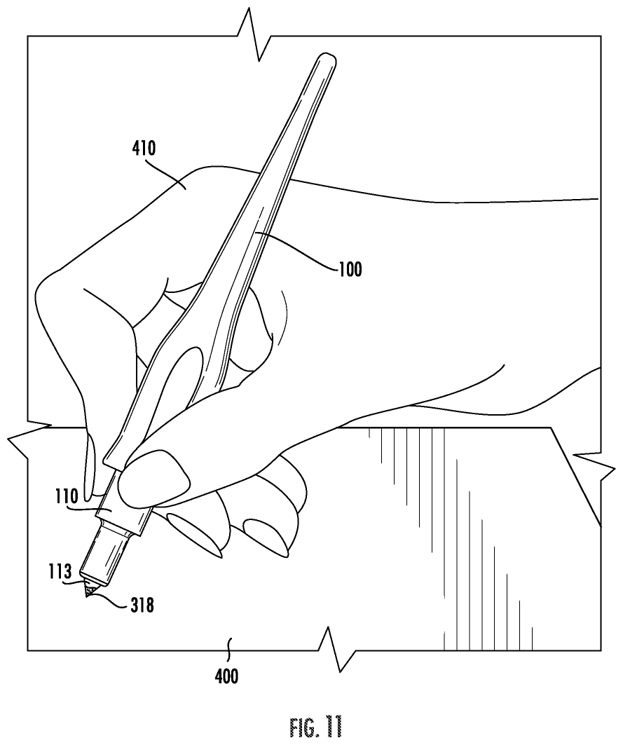

As shown in FIG. 6, the inkwell 332 may have a cavity 331 made for holding a volume of ink and a base 333 that may be configured to hold the inkwell 332 steady on a surface, such as a table surface 400. As shown in FIG. 7, an ink bottle 310 may hold a volume of ink within a container 316 sealed with a spigot 314 and a spigot cap 312. As shown in FIG. 8, a user may remove the spigot cap 312 from the spigot 314, and then may squeeze a volume of ink 318 into the cavity 331 of an inkwell 332 using their hand 410. As shown in FIG. 9, the user may couple the pen head 110 and the practice pen base 150 from the bag 300 of FIG. 5 to one another to form a practice tattoo pen 100. The practice tattoo pen 100 may have an exposed foam sharp tip 113. As shown in FIG. 10, the user may dip the exposed foam sharp tip 113 into the cavity 331 of the inkwell 332 to draw ink 318 into the exposed foam sharp tip 113. As shown in FIG. 11, the foam sharp tip 113 may absorb a volume of ink 318 from the inkwell 332, allowing a user to use the foam sharp tip 113 to apply ink to a surface.

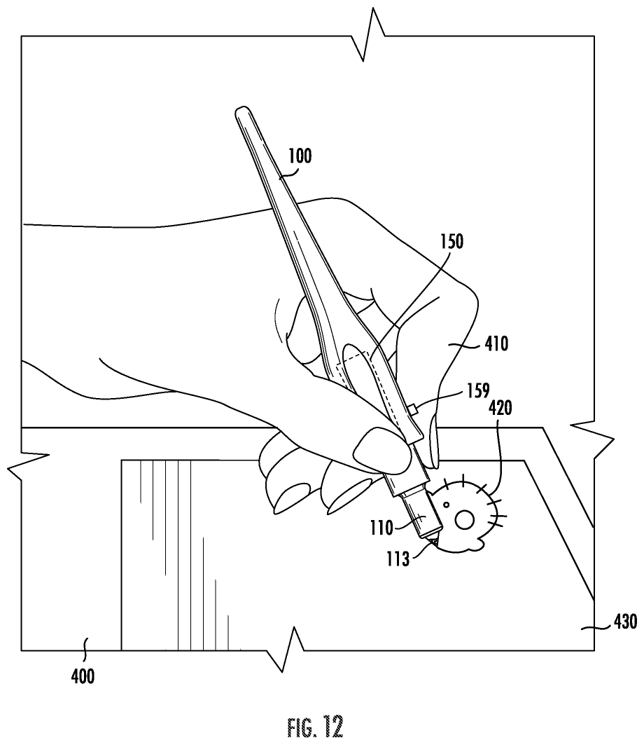

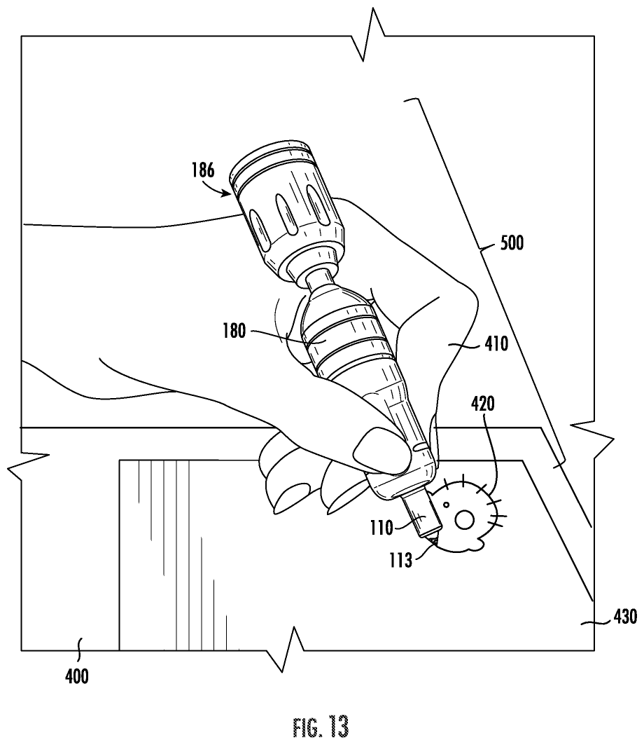

As shown in FIG. 12, a user may use the practice tattoo pen 100 to draw a stencil, such as the stencil 420, by brushing the foam sharp tip 113 across the paper surface 430 using their hand 410. If the user wishes to use other pen heads, such as the pen head 120 with a thick tip 123 or the pen head 130 with a brush tip 133 (see FIG. 3), the user may simply pull the pen head 110 out from the practice pen base 150, and insert another pen head into the cavity 152 of the pen practice pen base 150. Since the weight and the width of the practice tattoo pen base 150 of FIG. 3 may similar to the weight and the width of the mechanized pen base 180 of FIG. 4, the user builds muscle memory into their hand 410 while using the practice tattoo pen 100 as shown in FIG. 12. The user may also activate the vibrational motor 158 by pressing the button 159 to experience drawing a stencil while the practice tattoo pen 100 vibrates.

Later, as shown in FIG. 13, the user may use a mechanized tattoo pen 500 by coupling a pen head, such as the pen head 110, to the mechanized pen base 180, to apply ink to a stencil 420 on the paper surface 430. The user may use the muscle memory gained from using the practice tattoo pen 100 in FIG. 12 when the use the mechanized tattoo pen 500 of FIG. 13 to draw the stencil 420. The user may also remove the pen head 110 from the mechanized pen base 180 and apply another pen head to the mechanized pen base 180, such as the pen heads 120 and 130 of FIG. 4, or a needle cartridge to the mechanized pen base 180, such as the needle cartridge 160 of FIG. 4. The user may then turn on the motor 186 of the mechanized pen base 180 to repeatedly advance and retract a needle of the mechanized pen base 180 to apply ink to the skin of a user.

Various details of the practice and mechanized tattoo pens are explained in more detail below.

FIGS. 1-2 show an exemplary practice tattoo pen 100 that may be used to apply ink to a surface, such as a piece of paper or the skin of a person. The practice tattoo pen 100 may have a practice pen base 150 and a pen head 110. The pen head 110 may have a body 114 with a tip 113 (see FIG. 9) that is covered by the cap 112. When the cap 112 (see FIG. 1) is removed, the tip 113 (See FIG. 9) may be exposed with which a user can apply ink. As shown in FIG. 1, the practice pen base 150 may have a motor 158 located within the grip 153 that is configured to vibrate, which may help to simulate the feel of a motor in a mechanized tattoo pen when a user uses the practice tattoo pen 100. The motor 158 may be located within a volume of the grip 153, and may comprise an eccentric rotating mass that rotates an unbalanced mass to create a vibration similar to a mechanized tattoo pen. The motor 158 may be activated and deactivated via a user interface on the grip 153, such as the pop-up push button 159, which may be used to both activate and deactivate the motor 158. When a user pushes push button 159 when the motor 158 is off, the buttom 159 may close a circuit leading to the motor 158, activating the vibration feature. When the user pushes the push button 159 when the motor 158 is on, the button 159 may open the circuit leading to the motor 158, deactivating the vibration feature. The motor 158 may have a power source that may be charged via an induction charger also located within the grip 153. While a pop-up push button 159 is shown as being used to activate and deactivate the vibrational motor 158, any suitable mechanism may be used to activate or deactivate a motor of the practice tattoo pen 100. For example, the motor 158 may be activated or deactivated by rotating the body 114 of the pen head 110 relative to the grip 153 of the practice pen base 150, or a pop-up push button may be located within the cavity 152 (see FIG. 3) of the practice pen base 150, which may be activated or deactivated by applying pressure to the cap 112 (see FIG. 2) of the pen head 110.

As shown in FIG. 2, the grip 153 of the practice pen base 150 may have a plurality of ergonomic recesses located about the perimeter of the grip 153 to help a user to grip the practice pen base 150 during use. The practice pen base 150 may have a proximal end 151 and a distal end that tapers to a blunt tip 154. When the pen head 110 is coupled to the practice pen base 150, the body 114 of the pen head may abut the proximal end 151 of the practice pen base 150, as shown in FIG. 2. As shown in FIG. 3, the proximal end 151 of the practice pen base 150 may have a cavity 152 where the pen head 110 is shown as being detached from the practice pen base 150. The shaft 116 may be sized and disposed to mate with the cavity 152 and hold the practice pen base 150 and the pen head 110 to one another via a friction fit. While the practice pen base 150 is shown having a cavity 152 that is sized and disposed to receive the shaft 116 of the pen head 110, other connection means to join the practice pen base 150 and the pen head 110 may be used. For example the practice pen base 150 may have a projection that frictionally mates with a cavity of the pen head 110, or the shaft 116 of the pen head 110 may have outer threads that screw into matching threads of the cavity 152 of the practice pen base 150. When the shaft 116 is inserted into the cavity 152 of the practice pen base 150, the practice tattoo pen 110 may be used as a pen having a cap 112 (see FIG. 1) that covers the pointed tip 113 of FIG. 3 that extends from a body 114 of the pen head 110.

As shown in FIG. 3, the cavity 152 of the practice pen base 150 may be configured to mate with a plurality of pen heads. For example, each of the pen heads 110, 120, 130, and 176 shown in FIG. 3 may have a shaft 116, 126, 136, and 176, respectively, each sized and disposed to frictionally mate with the cavity 152. The shafts 116, 126, 136, and 176 and the cavity 152 and cavities may be designed to be friction-fit with one another, which allows for a head to be locked in place while applying ink to a surface, but also allows for a user to easily pull out a head and replace it with another head depending upon need.

Each of the pen heads 110, 120, 130, and 170 may have a tip that is configured to absorb a volume of ink. Each of the pen heads 110, 120, 130, and 170 may also be made of a different material and/or may be a different shape from the other pen heads. For example, the pen heads 110, 170 may have a sharpened tip 113, 173 comprising a porous foam that is configured to absorb ink through its pores. The pen head 120 may have a blunt tip 123 comprising a porous foam that is configured to absorb ink through its pores. The pen head 130 may have a brush tip 133 comprising a series of hairs or bristles that are configured to absorb ink through the fibers of the brush. Contemplated materials for pen heads comprise rubber, plastic, wool, cotton, or nylon. The materials of a pen head tip may be porous, which assists in ink absorption and release for when a user applies ink to a surface using the pen head tip. A user may dip a pen head tip in a volume of ink to absorb a volume of ink, and then may apply the pen head tip to a surface to apply ink to the surface, for example by drawing a stencil on the skin of a person.

A pen head may have a cap, such as cap 112 for pen head 110, that covers the tip of the head, such as the pen tip 113. The cap may prevent foreign matter, such as dirt or blood, from contaminating the tip of a head when the cap is disposed over the tip of the head. A cap may have a different size or shape to accommodate the tip of the head. For example, the caps 112, 122, and 172 for the pen heads 110, 120, and 170, respectively, may be shorter than the cap 132 for the pen head 130, as the brush tip 133 of the pen head 130 may be longer than the sharp tip 113 of the pen head 110 and the dull tip 123 of the pen head 120. While only four pen heads 110, 120, 130, and 170 are shown in FIG. 3, the proximal end 151 of the practice pen base 150 may be configured to mate with any number of pen heads and any number of needle cartridges or needle cartridges, allowing for flexibility in the interchangeability of heads with the practice pen base 150. A user may have a plurality of identical pen heads, such as pen heads 110 and 170, or may have a plurality of different pen heads, such as pen heads 110, 120, and 130, or a combination of identical and different pen heads. If a user does not wish to use a certain head, for example if a pen head becomes contaminated with a customer's blood, or if a user wishes to draw with a different shade of ink, the user may remove the head from the practice pen base 150, and replace it with a different head.

The proximal end 151 of the practice pen base 150 in FIG. 3 and the proximal end 181 of of the mechanized pen base 180 in FIG. 4 may each be configured to mate with a plurality of pen heads and needle cartridges, such as the pen heads 110, 120, 130, and 170 and the needle cartridges 140 and 160 of FIG. 4. In FIG. 3, the proximal end 151 of the practice pen base 150 may have a cavity 152 sized and disposed to receive each of the shafts 116, 126, 136, and 176 of the pen heads 110, 120, 130, and 170, respectively, and the shafts 146 and 166 of the needle cartridges 140 and 160, respectively, while in FIG. 4, the proximal end 181 of the mechanized pen base 180 may have a cavity 182 sized and disposed to receive each of the shafts 116, 126, 136, and 176 of the pen heads 110, 120, 130, and 170 respectively, and the shafts 146 and 166 of the needle cartridges 140 and 160, respectively. Such a configuration allows both the cavity 152 of the practice pen base 150 in FIG. 3 and the cavity 182 of the mechanized pen base 180 in FIG. 4 to fungibly accept any of the shafts 116, 126, 136, 176, 146, and 166 of the pen heads 110, 120, 130, and 170, and the needle cartridges 140 and 160, respectively. The practice pen base 150 of FIG. 3 may have a cavity 152 having a depth that only accepts the shafts of pen heads, such as the shafts 116, 126, 136, and 176 but not the shafts of needle cartridges, such as the shafts 146 and 166 of FIG. 4, or not the needle of the needle cartridges, such as the needle 168, while the mechanized pen base 180 of FIG. 4 may have a cavity 182 having a depth that accepts the shafts of both pen heads, such as the shafts 116, 126, and 136, and also the shafts of needle cartridges, such as the shafts 146 and 166.

The heads may be configured to be shaped to interchangeably mate with the proximal side of the practice pen base 150, as shown in FIG. 3, or the proximal side of the mechanized pen base 180, as shown in FIG. 4. Each of the pen heads 110, 120, 130, and 170 and the needle cartridges 140 and 160, has a shaft 116, 126, 136, 176, 146, and 166, respectively, which may mate with the cavity 182 in the mechanized pen base 180. The mechanized pen base 180 may have a grip 183 that has a size, shape, and weight similar to the size, shape, and weight of the grip 153 of the practice pen base 150 in FIG. 3. For example, both the mechanized pen base 180 and the practice pen base 150 may have a similar weight of at least 100 grams. Both the mechanized pen base 180 and the practice pen base 150 may have a similar width 185 and 155, respectively, of at least 2 cm.

The practice pen base 150 may have a grip 153 that is sized and weighted to be similar to the grip 183 of the mechanized pen base 180, shown in FIG. 4. Because the grip 153 of the practice pen base 150 in FIG. 3 is similar to the grip 183 of the mechanized pen base 180 of FIG. 4, a user that practices applying ink by using the grip 153 of the practice pen base 150 may develop a muscle memory that makes it easier to transition to using the grip 183 of the mechanized pen base 180 of FIG. 4. A user may hold the practice tattoo pen 100 using the grip 153 to manipulate the head, such as the pen head 110 shown in FIGS. 1 and 2, while applying ink to a surface. As shown in FIG. 3, the proximal end 151 of the practice pen base 150 may have a cavity 152 sized and disposed to accept a shaft of a head, such as the shaft 116 of the pen head 110.

As seen in FIG. 3, the proximal end 151 of the practice pen base 150 may also have a width 155 that is similar to the width 185 of the mechanized pen base shown in FIG. 4. The width 155 of the proximal end 151 of the practice pen base may be any suitable width, such as at least 2 cm, at least 3 cm, or at least 4 cm. The width 155 of the proximal end 151 of the practice pen base may be configured to be within 5 mm of the width 185 of the proximal end 151 of the mechanized pen base 180 shown in FIG. 4. The weight of the practice pen base 150 in FIG. 3 may also be similar to the weight of the mechanized pen base 180 in FIG. 4. For example, the weight of the practice pen base 150 may be configured to be within 10 grams of the weight of the mechanized pen base 180. The weight of the practice pen base 150 may be any suitable weight, such as at least 80 grams, 100 grams, or 120 grams.

The mechanized pen 180 may have an motor 186 which may be configured to vibrate, or may be configured to repeatedly advance and retract a needle, such as the needle 165 of the needle cartridge 160, to apply ink to a person's skin. The motor 186 of the mechanized pen 180 may have an actuator that couples to the distal end 168 of the needle 165, allowing for the actuator to repeatedly advance and retract the needle 165 of the needle cartridge 160 during use. A user who wishes to use the needle cartridge 160 may pour ink into the reservoir 167 via the ink inlet 169, which allows ink to dribble down to the tip of the needle 165 in the needle cartridge 160. The mechanized pen 180 may have a distal end 184 comprising a power inlet that allows a user to connect a power source, such as a battery or a power cable, to the mechanized pen 180 to provide power to the motor 186. The motor 186 may be activated and deactivated via a rotary switch that activates when the user turns the motor 186 in one direction relative to the grip 183 (e.g. clockwise), and deactivates when the user turns the motor 186 in an opposing direction relative to the grip 184 (e.g. counterclockwise). Activation of the motor may cause the motor to repeatedly advance and retract a needle of a needle cartridge or cartridge, such as the needle cartridges 140 and 160. While a rotary switch may be used to activate or deactivate the motor 186 of the mechanized pen 180, other methods of activating or deactivating the motor 186 may be used, for example a button or a switch.

While only three pen heads 110, 120, 130, and 170 and two needle cartridges 140 and 160, are shown in FIG. 4, the proximal end 181 of the mechanized pen base 180 may be configured to mate with any number of pen heads and any number of needle cartridges, allowing for flexibility in the interchangeability of heads with the mechanized pen base 180. If a user does not wish to use a certain head, for example if a pen head becomes contaminated with a customer's blood, or if a user wishes to draw with a different shade of ink, the user may remove the head from the mechanized pen base 180, and replace it with a different head having the same or a different pen tip. Since both the practice pen base 150 and the mechanized pen base 180 both share the ability to interchange heads, a user who practices applying ink with, and interchanging heads with, the practice pen base 150 may have few problems transitioning to applying ink with, and interchanging heads with, the mechanized pen base 180. While only one practice pen base 150 and one mechanized pen base 180 are shown here as being interchangeable with the pen heads 110, 120, and 130, and needle cartridges 140 and 160, the heads may be configured to mate with any number of practice pen bases and any number of mechanized pen bases.

Parts of a practice tattoo pen may be sold or otherwise provided to a user in a kit. For example, in FIG. 5, a plastic bag 300 may be provided containing a practice pen base 150, an ink bottle 310, a set of pen heads 110, 120, and 130, and a set of inkwells 332, 334, and 336. The plastic bag 300 contains elements that a user may use to quickly begin practicing with a practice pen, such as the practice pen 100 shown in FIGS. 1-2. The set of pen heads 110, 120, and 130 of FIG. 5 may interchangeably be coupled to the practice pen base 150, and the ink bottle 310 may be used to deposit ink into cavities of the set of inkwells 332, 334, and 336, respectively. A kit may contain any suitable combination of tattoo pen parts. For example, a kit may only contain a set of identical pen heads, such as the pen heads 110 and 170 of FIG. 3, or may contain a set of different pen heads, such as the pen heads 110, 120, and 130, or may contain a mixture of both same and different pen heads, such as the pen heads 110, 120, 130, and 170 or a mixture of both pen heads and needle cartridges, such as the pen heads 110, 120, 130, and 170 of FIG. 4 and the needle cartridges 140 and 170 of FIG. 4, or a set of pen heads, needle cartridges, and inkwells, such as the inkwells 332, 334, and 336, and one or more ink bottles, such as the ink bottle 310. A kit may of pen heads may comprise, for example, a satchel that holds a plurality of different pen tips that a user may use for the same customer. When the user needs to tattoo a different customer, the user may then just grab a different kit of pen heads that are all sanitary and are not contaminated by a customer's blood that may have wiped off on a tip of a pen head.

As shown in FIG. 6, an inkwell 332 may have a cavity 331 made for holding a volume of ink and a base 333 that holds the inkwell 332 steady on a surface, such as a table 400. The cavity 331 may be sized and configured to hold a volume of ink within the inkwell 332, such as at least 1 ml or at least 2 ml of ink. The cavity 331 of the inkwell 332 may have an opening that is sized and disposed to accept a pen tip, such as the pen tip 113 of the pen head 110 of FIG. 3, which allows a user to dip the pen tip into the cavity 331 of the inkwell 332 to absorb ink in the inkwell 332. The base 333 of the inkwell 332 may comprise a flat bottom to hold the inkwell 332 steady on a flat surface, or may be curved to hold the inkwell 332 steady on a curved surface. The inkwell 332 may be made of any suitable liquid-proof material, such as plastic, glass, or metal.

As shown in FIG. 7, an ink bottle 310 may hold a volume of ink within a container 316 sealed with a spigot 314 and a spigot cap 312. The ink bottle 310 may be configured to hold any suitable amount of ink, such as at least 30 ml or at least 50 ml of ink, within the container 316. The spigot 314 may be sealed to the container 316 using a threaded connection, and spigot cap 312 may be popped on and off of the container 316 to allow for a user to pour ink out of the ink bottle 310 via the spigot 314. The spigot cap 312 may provide an air-tight or a liquid-tight seal about an opening of the spigot 314 to ensure that any ink within the container 316 does not dry out when the spigot cap 312 is coupled to the spigot 314. The inkwell 332 may be made of any suitable liquid-proof material, such as plastic, glass, or metal.

As shown in FIG. 8, a user may place the inkwell 332 onto the table 400, and then may remove the spigot cap 312 from the spigot 314 to pour ink 318 from the ink bottle 310 into the cavity 331 of the inkwell 332. The container 316 of the ink bottle 310 may comprise a flexible material, such as a flexible plastic or rubber, which allows a user to squeeze the container 316 of the ink bottle 310 to help push ink 318 out of the spigot 314. Once the ink 318 has been poured into the inkwell 332, a user may closer the spigot 314 using the spigot cap 312 and get ready to use a practice tattoo pen, such as the practice tattoo pen 100 of FIGS. 1-2.

As shown in FIG. 9, a user may couple a pen head 110 to the practice pen base 150 to form a practice tattoo pen 100, and may remove the cap 112 from the pen head 110 to expose the sharp tip 113. The sharp tip 113 may be provided without any ink absorbed by the sharp tip 113, which allows a user to use the pen head 110 with any type of ink and any color of ink that the user may possess. The user may hold the practice tattoo pen 110 by the grip 153 using their hand 410, which should feel similar to the grip 183 of the mechanized pen base of FIG. 4. While the grip 153 may have a larger width 155 than a standard pen or pencil, as shown in FIG. 3, a user should be able to easily write with the practice tattoo pen 110 by squeezing the grip 153 using their thumb and forefinger.

As shown in FIG. 10, the user may dip the sharp tip 113 into the cavity 331 of the inkwell 332 to allow the exposed sharp top to absorb a volume of ink 318 from the inkwell 332. The sharp tip 113 may absorb any suitable volume of ink 318 to be able to apply ink to a surface by touching the surface with a portion of the sharp tip 113. For example, the sharp tip 113 may absorb at least 0.1, 0.2, or 0.3 ml of ink from the inkwell 332 when a user dips the sharp tip 113 into the inkwell 332. As shown in FIG. 11, the foam sharp tip may absorb a volume of ink 318 from the inkwell to allow a user to use the foam sharp tip 113 to apply ink to a surface. When a user abuts the sharp tip 113 to a surface, such as a paper or the skin of a person, ink absorbed by the sharp tip 113 may be wiped onto the surface, allowing a user to draw using the practice pen 100.

As shown in FIG. 12, a user may use the practice tattoo pen 100 to draw a stencil, such as the stencil 420, by brushing the foam sharp tip 113 across the surface 430 using their hand 410. If the user wishes to use other pen heads, such as the pen head 120 with a thick tip 123, the pen head 130 with a brush tip 133, the user may simply pull out the pen head 110 from the practice pen base 150, and insert another pen head into the cavity 152 of the pen practice pen base 150. Since the weight and the width of the practice tattoo pen 100 is similar to the weight and the width of the mechanized pen base 180, the user builds muscle memory into their hand 410 while using the practice tattoo pen 100.

As shown in FIG. 13, the user may also use a mechanized tattoo pen 500 by coupling a pen head, such as the pen head 110, to the mechanized pen base 180, to apply stenciling to the surface 430, or to apply a tattoo using a needle. A user using the mechanized tattoo pen 500 may apply a tattoo using the needle cartridge 160 of FIG. 4, and may accidentally wipe off a portion of a stencil while wiping blood off of a customer. The user may then remove the needle cartridge 160 from the mechanized tattoo pen 500, and couple the pen head 110 to the mechanized pen base 180. The user may then dip the pen head 110 into an inkwell to allow the sharp tip 113 of the pen head 110 to absorb ink, and then may redraw the stencil using the pen head 110. Once the user is satisfied, the user may then remove the pen head 110 and add a new needle cartridge to the mechanized tattoo pen to continue tattooing. If, during stenciling, the user gets blood onto the pen head 110, the user may then dispose of the pen head 110, and use a new pen head when the user needs to do more stenciling. Such a configuration allows a user to rapidly transition between tattooing using a needle tip and stenciling using a pen tip while using the same mechanized tattoo pen base 180, as the user does not need multiple tools, just multiple heads for the same tool.

The above description is given by way of example, and not limitation. Given the above disclosure, one skilled in the art could devise variations that are within the scope and spirit of the invention disclosed herein, including various ways of using various tattoo pens with interchangeable bases and heads. Further, the various features of the embodiments disclosed herein can be used alone, or in varying combinations with each other and are not intended to be limited to the specific combination described herein. Thus, the scope of the claims is not to be limited by the illustrated embodiments.

What is claimed is:

1. A practice tattoo pen for preparing to use a mechanized tattoo pen having a mechanized pen weight and a mechanized pen grip, comprising:
a practice pen base comprising a grip, a cavity, a practice pen weight, and a practice pen grip;
wherein the practice pen weight is within 10 grams of the mechanized pen weight;
wherein the practice pen grip has a width that is within 5 mm of a width of the mechanized pen grip; and
a first pen head comprising a first pen tip and a first pen shaft, wherein the first pen tip is configured to absorb a first volume of ink and the first pen shaft is configured to mate with the cavity of the practice pen base.

2. The practice tattoo pen of claim 1, wherein the practice pen weight comprises a weight of at least 100 grams.

3. The practice tattoo pen of claim 1, wherein the width of the practice pen grip comprises a width of at least 2 cm.

4. The tattoo pen of claim 1, wherein the first volume of ink comprises at least 0.2 ml of ink.

5. The practice tattoo pen of claim 1, further comprising a second pen head comprising a second pen tip and a second pen shaft, wherein the wherein the second pen tip is configured to absorb a second volume of ink and the second pen shaft is configured to mate with the cavity of the practice pen base.

6. The tattoo pen of claim 5, wherein the first pen tip comprises a porous foam, and wherein the second pen tip comprises a brush.

7. The tattoo pen of claim 5, wherein the first pen head, the second pen head, and a needle cartridge are each configured to mate with a cavity of the mechanized tattoo pen, and wherein a motor of the mechanized tattoo pen is configured to repeatedly advance and retract a needle tip of the needle cartridge.

8. A tattoo pen system, comprising:
a practice pen base comprising a practice pen grip and a cavity in the practice pen grip;
a mechanized pen base comprising a mechanized pen grip and a cavity the mechanized pen grip;
a first pen head comprising a first pen tip and a first pen shaft, wherein the first pen tip is configured to absorb a first volume of ink and the first pen shaft is configured to mate with the cavity of the practice pen base and the cavity of the mechanized pen base;
wherein the practice pen base has a weight that is within 10 grams of the mechanized pen weight; and
wherein the practice pen grip has a width that is within 5 mm of a width of the mechanized pen grip.

9. The tattoo pen system of claim 8, wherein the practice pen weight comprises a weight of at least 100 grams.

10. The tattoo pen system of claim 8, wherein the width of the practice pen grip comprises a width of at least 2 cm.

11. The tattoo pen system of claim 8, further comprising:
a first needle cartridge comprising a first needle tip and a first needle shaft, wherein the first needle shaft is configured to mate with the cavity of the mechanized pen base; and
wherein the mechanized pen base is configured to repeatedly advance and retract the first needle tip.

12. The tattoo pen system of claim 8, further comprising:
a second pen head comprising a second pen tip and a second pen shaft, wherein the second pen tip is configured to absorb a first volume of ink and the second pen shaft is configured to mate with the cavity of the practice pen base and the cavity of the mechanized pen base.

13. The tattoo pen system of claim 12, wherein the first pen tip comprises a porous foam, and wherein the second pen tip comprises a brush having a plurality of bristles.

14. A method of preparing to use a mechanized tattoo pen having a mechanized pen weight and a mechanized pen grip, comprising:
providing a practice pen base comprising a grip, a cavity, a practice pen weight within 10 grams of the mechanized pen weight, and a practice pen grip having a width that is within 5 mm of a width of the mechanized tattoo pen;
providing a first pen head comprising a first pen tip and a first pen shaft, wherein the first pen tip is configured to hold a first volume of ink, and wherein the first pen shaft is configured to mate with a cavity of the practice pen base;
coupling the first pen head to the cavity of the practice pen base; and
practicing applying ink to a surface using the first pen head by holding the grip of the practice pen base and pressing the first pen tip down on the surface.

15. The method of claim 14, further comprising:
applying ink to a stencil on a skin surface using a needle of the mechanized tattoo pen;
redrawing the stencil using the first pen head by holding the grip of the practice pen base and pressing the first pen tip down to the skin surface.

16. The method of claim 15, further comprising activating a motor of the mechanized tattoo pen to repeatedly advance and retract the needle.

17. The method of claim 14, further comprising:
applying ink to a stencil on a skin surface using a needle of the mechanized tattoo pen;
removing a needle cartridge from a pen base of the mechanized tattoo pen, wherein the needle cartridge comprises a needle;
removing the first pen head from the cavity of the practice pen base;
coupling the first pen head to a cavity of the mechanized tattoo pen; and
redrawing the stencil using the first pen head by holding the mechanized pen grip and pressing the first pen tip down to the skin surface.

18. The method of claim 14, further comprising moving the grip of the practice pen base to dip the first pen tip of the first pen head into an inkwell to absorb ink from the inkwell.

19. The method of claim 14, further comprising:
removing the first pen head from the cavity of the practice pen base;
coupling a second pen head to the cavity of the practice pen base;
practicing applying ink to the surface using the second pen head by holding the grip of the practice pen base and pressing the second pen tip down on the surface.

20. The method of claim 19, wherein the first pen head comprises a porous foam tip and the second pen head comprises a brush tip.
